(19) United States
(12) Patent Application Publication
(10) Pub. No.: US 2017/0036044 A1
(43) Pub. Date: Feb. 9, 2017

(54) ULTRASONIC TRANSDUCER AND ULTRASONIC MEDICAL DEVICE

(71) Applicant: OLYMPUS CORPORATION, Tokyo (JP)

(72) Inventor: Hiroshi ITO, Tokyo (JP)

(73) Assignee: OLYMPUS CORPORATION, Tokyo (JP)

(21) Appl. No.: 15/299,687

(22) Filed: Oct. 21, 2016

Related U.S. Application Data
(63) Continuation of application No. PCT/JP2015/051783, filed on Jan. 23, 2015.

(30) Foreign Application Priority Data
Apr. 25, 2014 (JP) 2014-091437

(51) Int. Cl. A61N 7/00; B06B 1/06; H01L 41/083

(52) U.S. Cl. CPC A61N 7/00; H01L 41/083; B06B 1/0611

(57) ABSTRACT

An ultrasonic transducer 1 includes two metal blocks 2, a plurality of piezoelectric elements 3 stacked between the metal blocks 2, a bonding material 4 bonding the metal blocks 2 and the piezoelectric elements 3, and the piezoelectric elements 3 to each other, and a heterogeneous material part 5 having a thermal expansion coefficient differed from a thermal expansion coefficient of the metal block 2 and provided in a notch part 2b formed at an end portion of the metal block 2 on a bonding plane side with respect to the piezoelectric element 3.

### CROSS-REFERENCE TO RELATED APPLICATIONS

This application is a continuation claiming priority on the basis of Japan Patent Application No. 2014-038277 applied in Japan on Feb. 28, 2014 and based on PCT/JP2015/053452 filed on Feb. 9, 2015. The contents of both the PCT application and the Japan Application are incorporated herein by reference.

### BACKGROUND OF THE INVENTION AND RELATED ART STATEMENT

The present invention relates to an ultrasonic transducer that excites ultrasonic vibrations and an ultrasonic medical device.

There is known, as an ultrasonic transducer, one called Langevin transducer having a structure in which a piezoelectric transducer such as a piezoceramic is held between metal blocks and all parts are combined. The Langevin transducer is an element that vibrates the entire element at the natural frequency of the entire element by utilizing a resonance phenomenon of the metal block and thereby can generate efficient ultrasonic vibration. In general, the Langevin transducer has a structure in which the piezoelectric transducer and metal block are combined together by adhesive bonding or bolt clamping.

However, when the piezoelectric element and metal block are combined together by a brazing material such as a solder in order to efficiently transmit vibration, a process of heating a bonding portion to a high temperature is required. Then, since the piezoelectric element and metal block have different thermal expansion coefficients, a stress occurs at the bonding portion.

The following describes stress relaxation relating to the present embodiment using a reference example.

FIGS. 11A to 11D illustrate a reference example of a bonding portion between a metal block 102 and a piezoelectric element 103 of an ultrasonic transducer 101. FIG. 11A illustrates a state of the metal block 102 and piezoelectric element 103 upon a high-temperature bonding process where a solder as a bonding material 104 is melted. FIG. 11B illustrates a tentative state of the metal block 102 and piezoelectric element 103 upon cooling after a bonding process where they are not actually bonded together. FIG. 11C illustrates an actual state of the metal block 102 and piezoelectric element 103 upon cooling after a bonding process. FIG. 11D illustrates a case where both the metal block 102 and piezoelectric element 103 are deformed. Although a plurality of piezoelectric elements 103 are actually stacked, only one piezoelectric element 103 is illustrated in FIGS. 11A to 11D for simplicity of explanation.

In the reference example of FIGS. 11A to 11D, a case where a thermal expansion coefficient α3 of the piezoelectric element 103 is smaller than a thermal expansion coefficient α2 of the metal block 102 will be described. As illustrated in FIG. 11A, upon the high-temperature bonding process, it is assumed that the bonding material 104 is melted and that no stress acts on the piezoelectric element 103 and metal block 102.

In the state of FIG. 11A, the piezoelectric element 103 and metal block 102, which are not bonded together, are cooled to a room temperature. Then, since the thermal expansion coefficient α3 of the piezoelectric element 103 is smaller than the thermal expansion coefficient α2 of the metal block 102, shrinkage of the piezoelectric element 103 is smaller than that of the metal block 102. That is, shrinkage of the metal block 102 is larger than that of the piezoelectric element 103.

The piezoelectric element 103 and metal block 102 are actually bonded together, so that a compression stress occurs in the piezoelectric element 103 having the small thermal expansion coefficient α3, and a tensile stress occurs in the metal block 102 having the large thermal expansion coefficient α2. When there is an appropriate balance between the stresses, the profiles of the piezoelectric element 103 and metal block 102 become proportional as illustrated in FIG. 11C.

However, when considering a three-dimensional stress, the metal block 102 is shrunk more than the piezoelectric element 103 also in a thickness direction, so that, as illustrated in FIG. 11D, the piezoelectric element 103 may be deformed so as to be pulled by the metal block 102 on an outer peripheral side thereof.

In order to cope with this problem, an ultrasonic transducer is disclosed, in which lattice-shaped grooves or a plurality of recesses are formed on a bonding plane of each of the metal blocks to be bonded, by means of an adhesive, to an electrode provided on both upper and lower surfaces of the piezoelectric transducer to reduce shearing strain generated during driving or a dielectric loss on the bonding plane, to thereby reduce a temperature rise during driving to prevent a crack in the piezoelectric transducer and to thereby stabilize a vibration mode (see JP 2008-128875A).

### SUMMARY OF INVENTION

An ultrasonic transducer according an aspect of the present invention includes: two metal blocks; a plurality of piezoelectric elements stacked between the metal blocks; a bonding material bonding the metal blocks and the piezoelectric elements, and the piezoelectric elements to each other, and a heterogeneous material part provided in a notch part formed in a bonding plane of the metal block with respect to the piezoelectric element and having a thermal expansion coefficient different from that of the metal block.

An ultrasonic medical device according to another aspect of the present invention includes: the ultrasonic transducer described above; and a probe distal end part receiving ultrasonic vibration generated in the ultrasonic transducer and treating the body tissue.

### BRIEF DESCRIPTION OF DRAWINGS

FIGS. 1A and 1B illustrate an ultrasonic transducer according to the present embodiment;

FIGS. 2A and 2B illustrate a metal block of the ultrasonic transducer according to a first embodiment;

FIG. 3 illustrates a bonding portion between the metal block and a piezoelectric element of the ultrasonic transducer according to the first embodiment;

FIGS. 4A and 4B illustrate an example of a shape of a heterogeneous material part of the ultrasonic transducer according to the first embodiment;

FIGS. 5A and 5B illustrate the metal block of the ultrasonic transducer according to a second embodiment;

FIG. 6 illustrates the bonding portion between the metal block and piezoelectric element of the ultrasonic transducer according to the second embodiment;

FIGS. 7A and 7B illustrate an example of a shape of the heterogeneous material part of the ultrasonic transducer according to the second embodiment;

FIG. 8 illustrates an entire configuration of an ultrasonic medical device according to the present embodiment;

FIG. 9 illustrates a schematic entire configuration of a transducer unit of the ultrasonic medical device according to the present embodiment;

FIG. 10 illustrates an entire configuration of an ultrasonic medical device according to another aspect of the ultrasonic medical device according to the present embodiment; and

FIGS. 11A to 11D illustrate a reference example of a bonding portion between a metal block and a piezoelectric element.

### DESCRIPTION OF EMBODIMENTS

Hereinafter, preferred embodiments of an ultrasonic transducer 1 according to the present invention will be described.

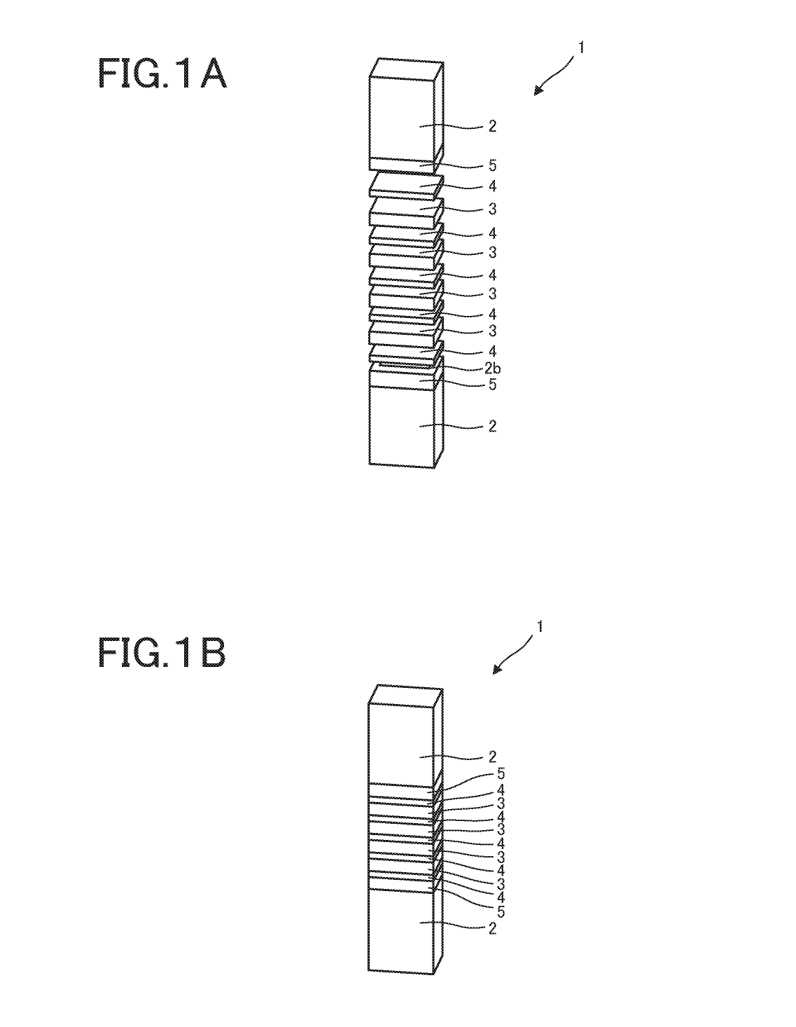

FIGS. 1A and 1B illustrate an ultrasonic transducer 1 according to the present embodiment. FIG. 1A illustrates the ultrasonic transducer 1 before bonding. FIG. 1B illustrates the ultrasonic transducer 1 after bonding.

As illustrated in FIG. 1A, the ultrasonic transducer 1 of the present embodiment includes two metal blocks 2, a plurality of piezoelectric elements 3 stacked between the metal blocks 2, a bonding material 4 bonding the metal blocks 2 and the piezoelectric elements 3, and the piezoelectric elements 3 to each other, and a heterogeneous material part 5 having a thermal expansion coefficient different from that of the metal block 2 and provided in a notch part 2b formed at an end portion of the metal block 2 on a bonding plane side with respect to the piezoelectric element 3.

The metal block 2 and piezoelectric element 3, and the piezoelectric elements 3 are bonded in a close contact state by the bonding material 4 as illustrated in FIG. 1B. The bonding is achieved by heating to a temperature at which the bonding material 4 is melted and then cooling.

Materials of the ultrasonic transducer 1 according to the present embodiment will be described.

Single-crystal lithium niobate having a high Curie point is used for the piezoelectric element 3. For example, preferably a lithium niobate wafer having a crystal orientation called 36-degree rotation Y cut is used so as to make large an electro-mechanical coupling coefficient in a thickness direction of the piezoelectric element 3 Then, a base metal such as Ti/Pt or Cr/Ni/Au is formed on both front and back surfaces of the lithium niobate wafer so as to improve wettability and adhesion between the lithium niobate wafer and a lead-free solder, followed by, e.g., dicing into rectangular pieces. A lead-free solder having a melting point lower than the Curie point, preferably, a melting point equal to or lower than half of the Curie point is used for the bonding material 4. However, when the solder is used as the bonding material and supplied in the form of solder pellets, it is difficult to bond a portion having an irregular shape without bubbles being generated. Thus, the bonding portions between the piezoelectric element 3 and metal block 2 and between the piezoelectric element 3 and heterogeneous material part 5 preferably have flat surfaces.

The metal block 2 and heterogeneous material part 5 are formed of materials having different thermal expansion coefficients selected respectively from among an aluminum alloy such as duralumin, a titanium alloy such as 64Ti, pure titanium, stainless steel, soft steel, nickel-chrome steel, tool steel, brass, and monel metal.

The ultrasonic transducer 1 formed as illustrated in FIG. 1B is attached, at its side, to a flexible board connected to an unillustrated electric cable. Further, like general ultrasonic transducers, positive and negative electrode layers are alternately attached to both ends and between the stacked piezoelectric elements 3. Application of a driving electric signal to the piezoelectric elements 3 allows the ultrasonic transducer 1 to be driven.

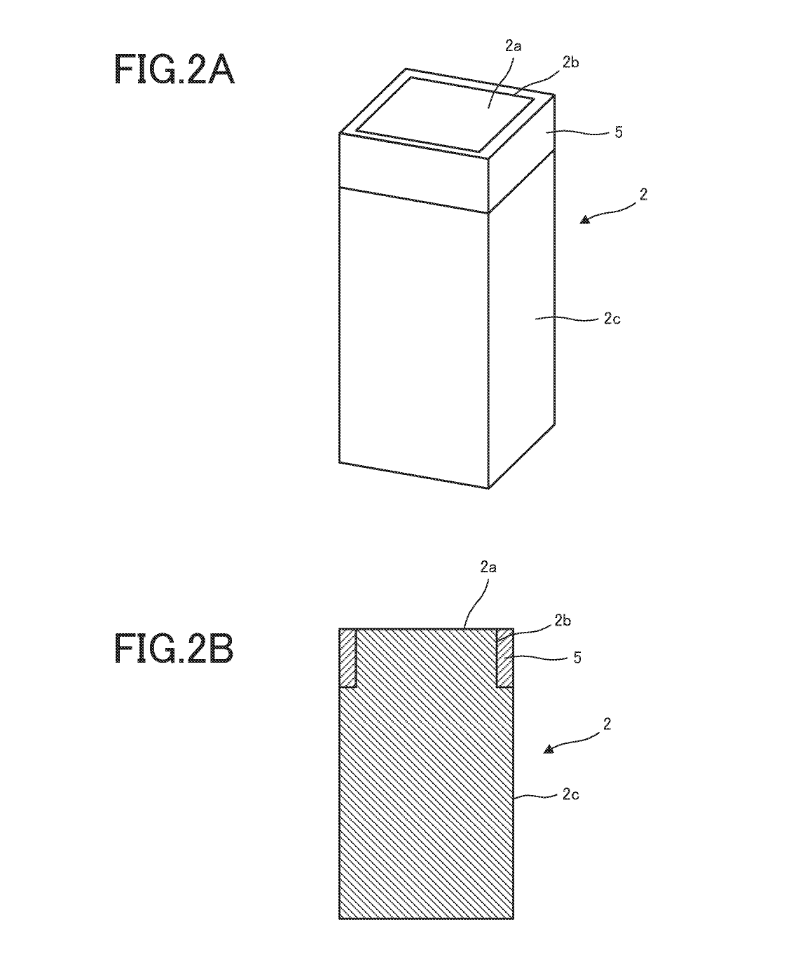

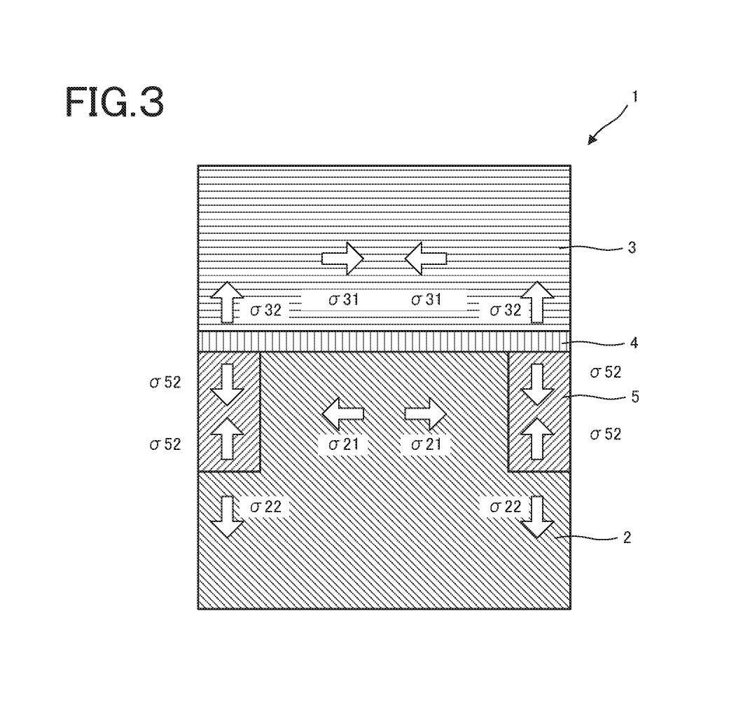

FIGS. 2A and 2B illustrate the metal block 2 and heterogeneous material part 5 of the ultrasonic transducer 1 according to a first embodiment. FIG. 2A is a perspective view of the metal block 2 and heterogeneous material part 5. FIG. 2B is a cross-sectional view of the metal block 2 and heterogeneous material part 5. FIG. 3 illustrates the bonding portion between the metal block 2 and piezoelectric element 3 of the ultrasonic transducer 1 according to the first embodiment.

The metal block 2 of the ultrasonic transducer 1 according to the first embodiment has a notch part 2b which is formed at an end portion of the metal block 2 on a bonding plane 2a side with respect to the piezoelectric element 3 illustrated in FIGS. 1A and 1B. The bonding plane 2a preferably has a flat surface. As illustrated in FIGS. 2A and 2B, the notch part 2b of the first embodiment is a part obtained by scraping an outer surface 2c of the metal block 2 inward.

In the notch part 2b, a heterogeneous material part 5 having a thermal expansion coefficient different from that of the metal block 2 is provided. The heterogeneous material part 5 according to the first embodiment is preferably flush with or substantially flush with the bonding plane 2a and outer surface 2c of the metal block 2. A dimension of the heterogeneous material part 5 may be appropriately determined according to a material to be used therefor.

In the ultrasonic transducer 1 according to the first embodiment, materials of the respective members are preferably determined so that a thermal expansion coefficient α2 of the metal block 2, a thermal expansion coefficient α3 of the piezoelectric element 3, and a thermal expansion coefficient α5 of the heterogeneous material part 5 satisfy at least a relationship of 60 5<α2 and, more preferably, α5<α3<α2.

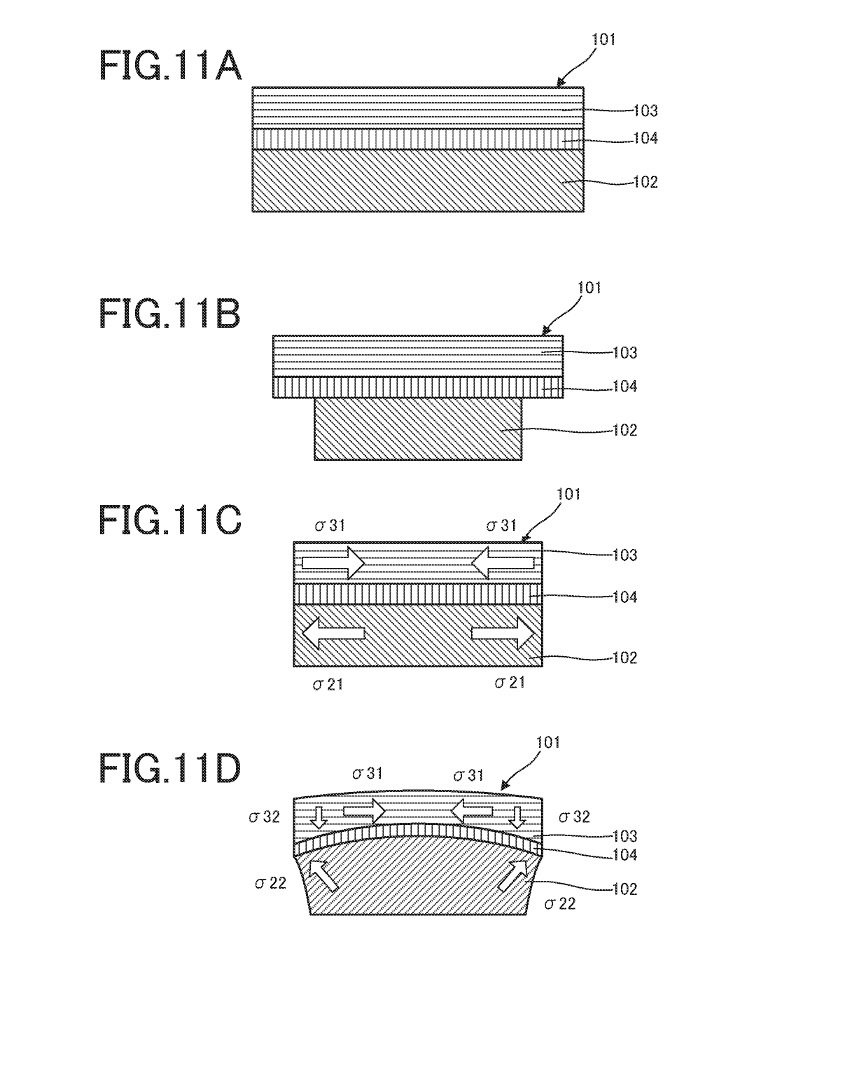

As illustrated in FIG. 3, in the ultrasonic transducer 1 according to the first embodiment, assuming that a stress acting inside the metal block 2 in the bonding plane direction is σ21, a stress acting inside the piezoelectric element 3 in the bonding plane direction is σ31, a stress acting near an outer periphery of the metal block 2 in a thickness direction is σ22, a stress acting near an outer periphery of the piezoelectric element 3 in a thickness direction is σ32, and a stress acting on the heterogeneous material part 5 in a thickness direction is σ52, the stress σ21 acting inside the metal block 2 and stress σ31 acting inside the piezoelectric element 3 that occur during a cooling process from a melting temperature of the bonding material 4 to a room temperature can be reduced by the heterogeneous material part 5. Further, providing the heterogeneous material part 5 around the metal block 2 allows the stress σ32 acting on the outer periphery of the piezoelectric element 3 in the thickness direction to be reduced. Further, depending on a configuration of the heterogeneous material part 5, it is possible to make the stress σ32 acting on the outer periphery of the piezoelectric element 3 be a compression stress acting in an opposite direction to that illustrated in FIG. 11D.

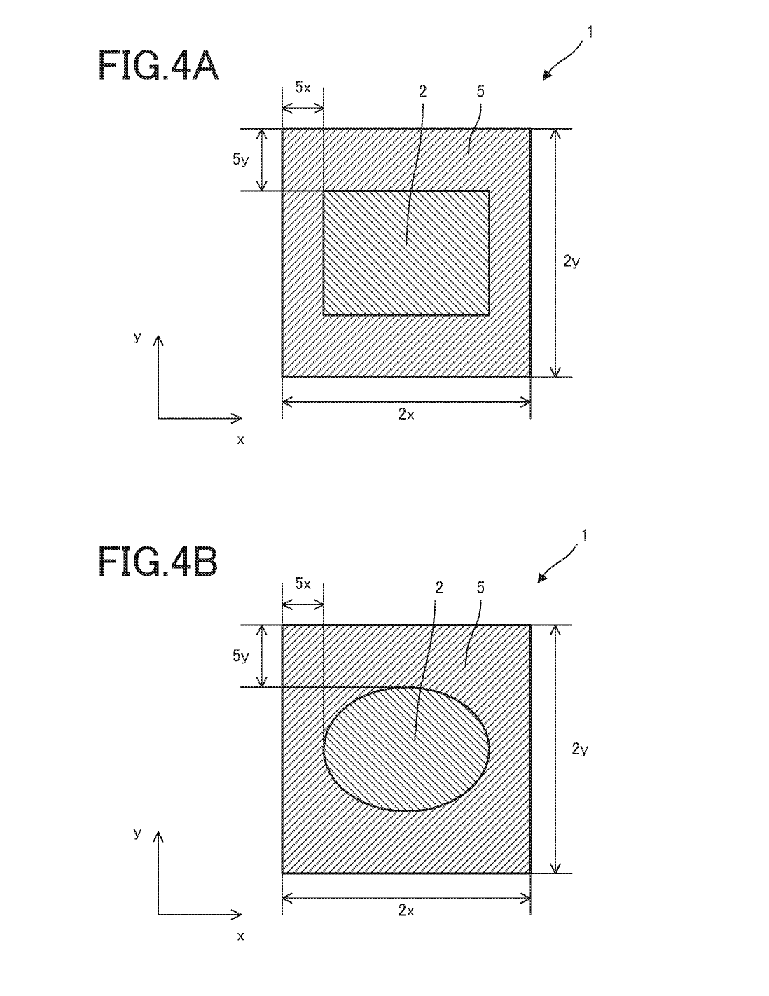

FIGS. 4A and 4B illustrate an example of a shape of the bonding plane of the heterogeneous material part 5 of the ultrasonic transducer 1 according to the first embodiment. FIG. 4A illustrates an example of a shape of the bonding plane of the heterogeneous material part 5. FIG. 4B illustrates another example of a shape of the heterogeneous material part 5.

The thermal expansion coefficient α3 of the piezoelectric element 3 to be used in the present embodiment shows anisotropy in an in-plane direction since the piezoelectric element 3 is monocrystalline. For example, in the first embodiment, assuming that a thermal expansion coefficient of the piezoelectric element 3 in FIGS. 4A and 4B in an x-direction is α3x and a thermal expansion coefficient thereof in a y-direction is α3y, a relationship of α3x>α3y is satisfied. Further, a thermal expansion coefficient of the metal block 2 is assumed to be α2, and a thermal expansion coefficient of the heterogeneous material part 5 is assumed to be α5.

In the example of FIG. 4A, a difference between the thermal expansion coefficient α2 of the metal block 2 and the thermal expansion coefficient α3y of the piezoelectric element 3 in the in-plane direction is larger than a difference between the thermal expansion coefficient α2 of the metal block 2 and the thermal expansion coefficient α3x of the piezoelectric element 3. That is, a difference between the thermal expansion coefficients of the metal block 2 and piezoelectric element 3 is larger in the y-direction than in the x-direction. As a result, a thermal stress larger than that in the x-direction occurs in the y-direction. Thus, by making larger a ratio 5y/2y of a dimension 5y of the heterogeneous material part 5 in the y-direction to a dimension 2y of the metal block 2 in the y-direction than a ratio 5x/2x of a dimension 5x of the heterogeneous material part 5 in the x-direction to a dimension 2x of the metal block 2 in the x-direction, effect of the stress relaxation in the y-direction can be enhanced.

Further, as the example illustrated in FIG. 4B, even when the outer shape and inner shape of the heterogeneous material part 5 are different, by making larger a ratio 5y/2y of a dimension 5y of the heterogeneous material part 5 in the y-direction to a dimension 2y of the metal block 2 in the y-direction than a ratio 5x/2x of a dimension 5x of the heterogeneous material part 5 in the x-direction to a dimension 2x of the metal block 2 in the x-direction, effect of the stress relaxation in the y-direction can be enhanced.

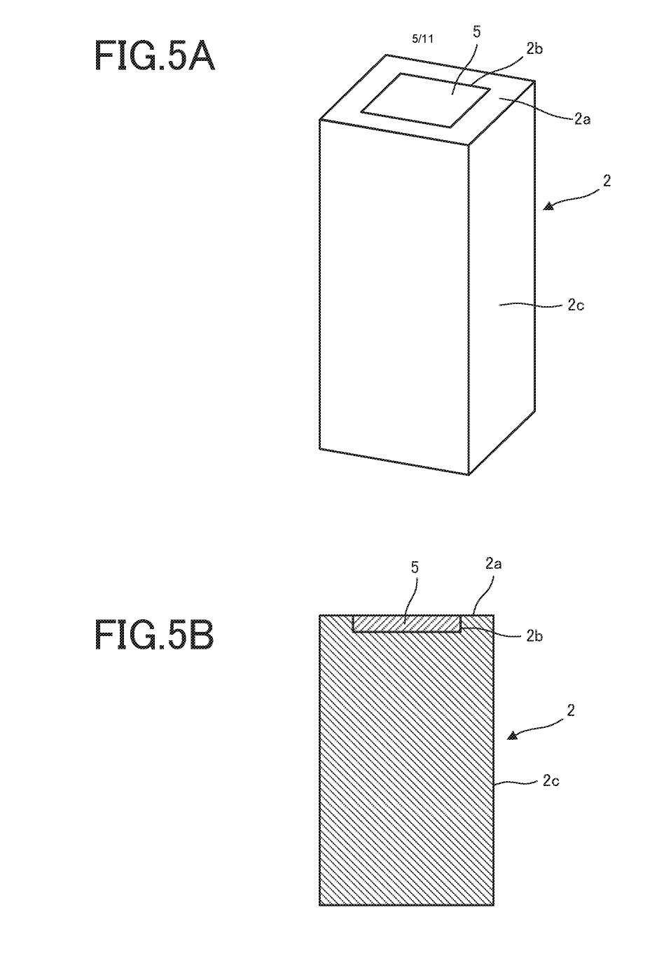

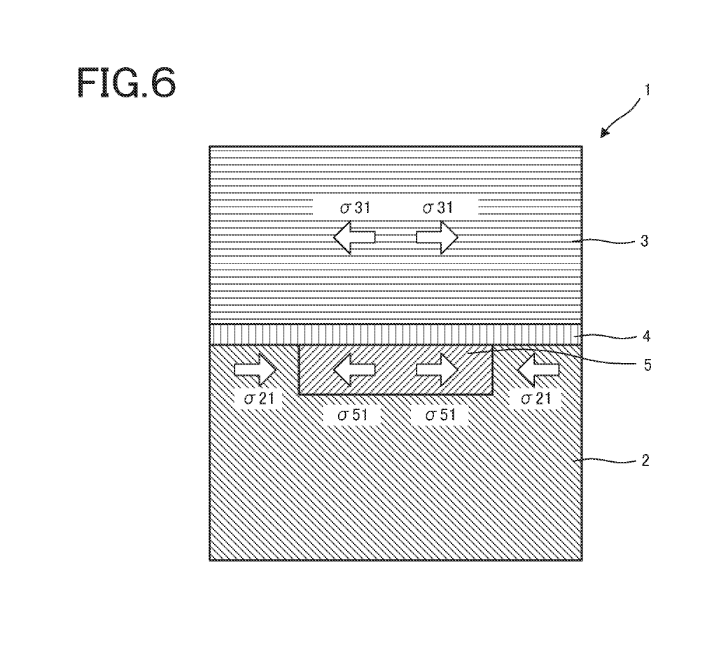

FIGS. 5A and 5B illustrate the metal block 2 and heterogeneous material part 5 of the ultrasonic transducer 1 according to a second embodiment. FIG. 5A is a perspective view of the metal block 2 and heterogeneous material part 5. FIG. 5B is a cross-sectional view of the metal block 2 and heterogeneous material part 5. FIG. 6 illustrates the bonding portion between the metal block 2 and piezoelectric element 3 of the ultrasonic transducer 1 according to the second embodiment.

The metal block 2 of the ultrasonic transducer 1 according to the second embodiment has a notch part 2b which is formed at an end portion of the metal block 2 on the bonding plane 2a side with respect to the piezoelectric element 3 illustrated in FIGS. 1A and 1B. The bonding plane 2a preferably has a flat surface. As illustrated in FIGS. 5A and 5B, the notch part 2b of the second embodiment is a part obtained by scraping the metal block 2 inward.

In the notch part 2b, a heterogeneous material part 5 having a thermal expansion coefficient different from that of the metal block 2 is provided. The heterogeneous material part 5 according to the second embodiment is preferably flush with or substantially flush with the bonding plane 2a of the metal block 2. A dimension of the heterogeneous material part 5 may be appropriately determined according to a material to be used therefor.

In the ultrasonic transducer 1 according to the second embodiment, materials of the respective members are preferably determined so that a thermal expansion coefficient α2 of the metal block 2, a thermal expansion coefficient α3 of the piezoelectric element 3, and a thermal expansion coefficient α5 of the heterogeneous material part 5 satisfy at least a relationship of α2<α5 and, more preferably, α2<α3<α5.

A cooling process from a melting temperature of the bonding material 4 to a room temperature is considered assuming that a stress acting on the metal block 2 in the bonding plane direction is σ21, a stress acting on the piezoelectric element 3 in the bonding plane direction is σ31, and a stress acting on the heterogeneous material part 5 in the bonding plane direction is σ52. In this case, shrinkage of the piezoelectric element 3 is larger than that of the metal block 2, so that a tensile stress acts on the piezoelectric element 3 in the bonding plane direction. However, in the ultrasonic transducer 1 according to the second embodiment, the heterogeneous material part 5 has a large thermal expansion coefficient, and shrinkage of the metal block in the bonding plane direction becomes the sum of shrinkage of the metal block 2 and that of the heterogeneous material part 5. As a result, shrinkage of the metal block 2 in the bonding plane direction becomes close to shrinkage of the piezoelectric element 3. Thus, the stress acting on the metal block 2 and piezoelectric element 3 that occurs during the cooling process from a melting temperature of the bonding material 4 to a room temperature can be reduced by the heterogeneous material part 5.

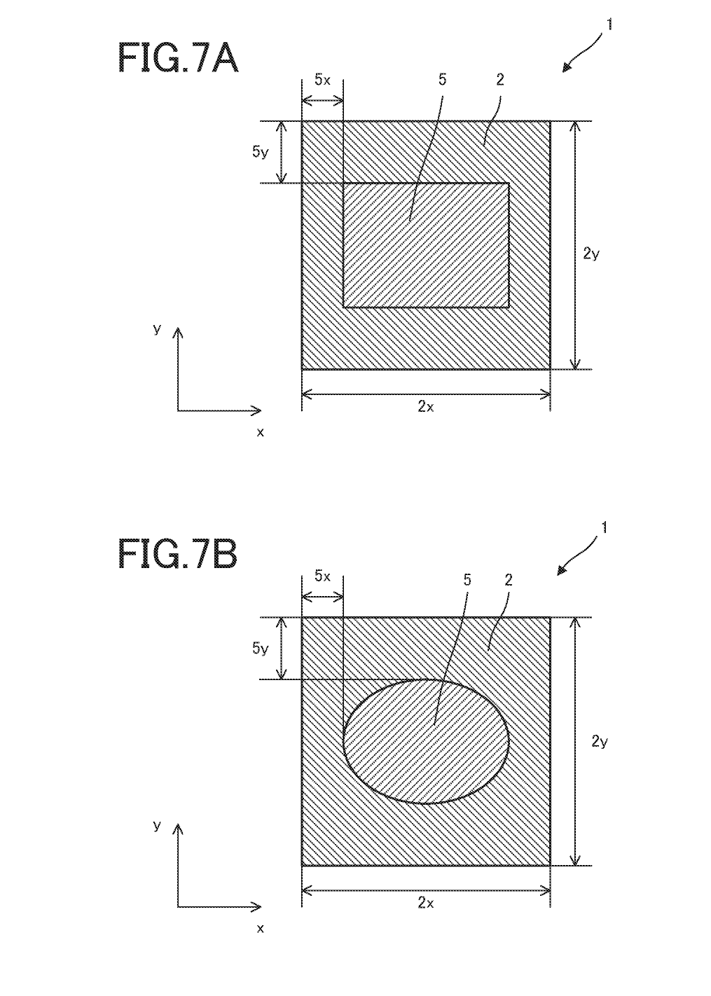

FIGS. 7A and 7B illustrate an example of a shape of the bonding plane of the heterogeneous material part 5 of the ultrasonic transducer 1 according to the second embodiment. FIG. 7A illustrates an example of a shape of the bonding plane of the heterogeneous material part 5. FIG. 7B illustrates another example of a shape of the bonding plane of the heterogeneous material part 5.

The thermal expansion coefficient α3 of the piezoelectric element 3 to be used in the present embodiment shows anisotropy in an in-plane direction since the piezoelectric element 3 is monocrystalline. For example, in the second embodiment, assuming that a thermal expansion coefficient of the piezoelectric element 3 in FIGS. 7A and 7B in the x-direction is α3x and a thermal expansion coefficient thereof in the y-direction is αy, a relationship of α3x>α3y is satisfied. Further, a thermal expansion coefficient of the metal block 2 is assumed to be α2, and a thermal expansion coefficient of the heterogeneous material part 5 is assumed to be α5.

In the example of FIG. 7A, a difference between the thermal expansion coefficient α2 of the metal block 2 and the thermal expansion coefficient α3y of the piezoelectric element 3 in the in-plane direction is larger than a difference between the thermal expansion coefficient α2 of the metal block 2 and the thermal expansion coefficient α3x of the piezoelectric element 3 in the in-plane direction. That is, a difference between the thermal expansion coefficients of the metal block 2 and piezoelectric element 3 is larger in the y-direction than in the x-direction. As a result, a thermal stress larger than that in the x-direction occurs in the y-direction. Thus, by making a relationship among X- and y-direction-dimensions 5x and 5y from the outer periphery of the metal block 2 to the heterogeneous material part 5 and x- and y-direction-outer diameters 2x and 2y of the metal block 2 satisfy (5x/2x)<(5y/2y), effect of the stress relaxation in the x-direction can be enhanced.

Further, as the example illustrated in FIG. 7B, even when an outer shape and an inner shape of the heterogeneous material part 5 are different, a thermal stress larger than that in the y-direction occurs in the x-direction. Thus, by making a relationship among X-and y-direction-dimensions 5x and 5y from the outer periphery of the metal block 2 to the heterogeneous material part 5 and x- and y-direction-outer diameters 2x and 2y of the metal block 2 satisfy (5x/2x)<(5y/2y), effect of the stress relaxation in the x-direction can be enhanced.

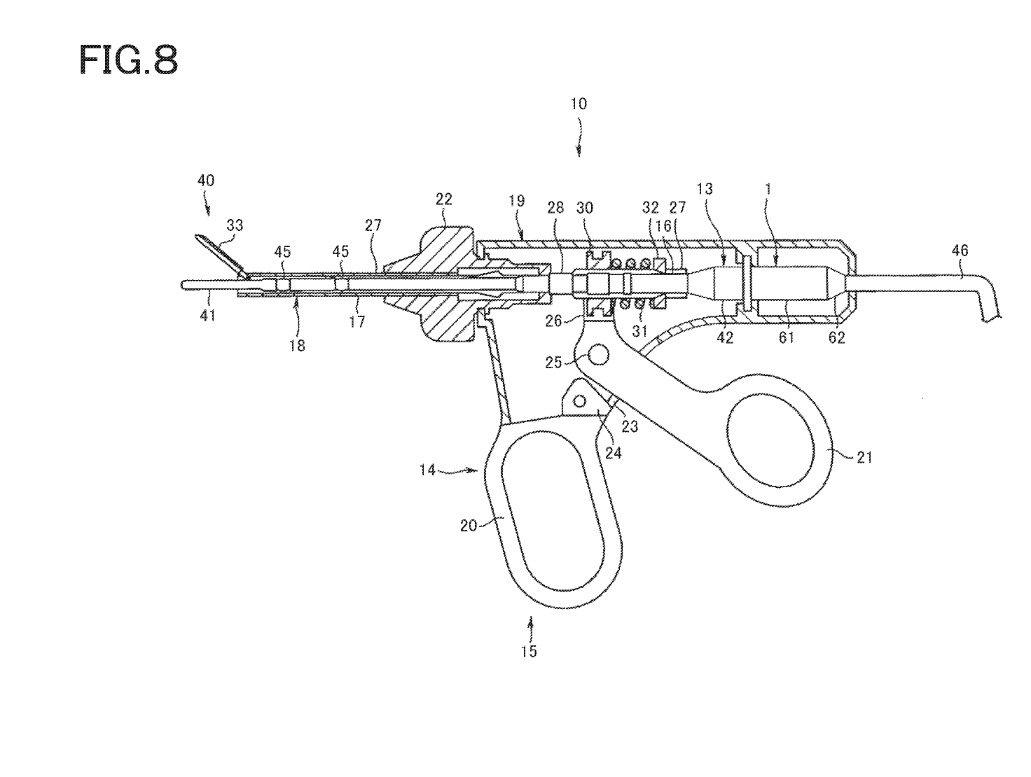

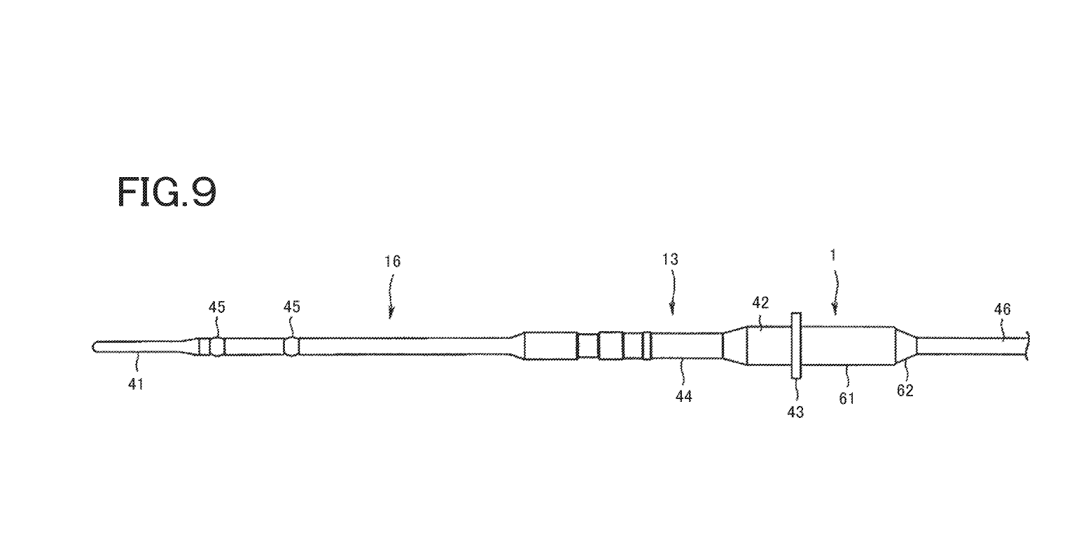

FIG. 8 illustrates an entire configuration of an ultrasonic medical device according to the present embodiment. FIG. 9 illustrates a schematic entire configuration of a transducer unit of the ultrasonic medical device according to the present embodiment.

An ultrasonic medical device 10 illustrated in FIG. 8 includes a transducer unit 13 having an ultrasonic transducer 1 that mainly generates ultrasonic vibration and a handle unit 14 for an operator to treat an affected part using the ultrasonic vibration.

The handle unit 14 includes an operation part 15, an insertion sheath part 18 constituted of a long outer tube 17, and a distal end treatment part 40. A base end portion of the insertion sheath part 18 is attached to the operation part 15 so as to be rotatable about an axis of the sheath part 18. The distal end treatment part 40 is provided at a distal end of the insertion sheath part 18. The operation part 15 of the handle unit 14 includes an operation part main body 19, a fixed handle 20, a movable handle 21, and a rotary knob 22. The operation part main body 19 is formed integrally with the fixed handle 20.

A slit 23 through which the movable handle 21 is inserted is formed on a back side of a connection portion between the operation part main body 19 and fixed handle 20. An upper portion of the movable handle 21 is inserted through the slit 23 and extends inside the operation part main body 19. A handle stopper 24 is fixed to a lower end portion of the slit 23. The movable handle 21 is turnably attached to the operation part main body 19 through a handle spindle 25. Accompanying a turning movement of the movable handle 21 with the handle spindle 25 as a center, the movable handle 21 is opened/closed with respect to the fixed handle 20.

A substantially U-shaped connection arm 26 is provided at an upper end portion of the movable handle 21. The insertion sheath part 18 has an outer tube 17 and an operation pipe 27 inserted into the outer tube 17 so as to be movable in an axial direction of the outer tube 17. A large diameter portion 28 having a diameter larger than that of a distal end side portion is formed at a base end portion of the outer tube 17. The rotary knob 22 is fitted around the large diameter portion 28.

A ring-shaped slider 30 is provided on an outer peripheral surface of the operation pipe 27 so as to be movable in an axial direction of the operation pipe 27. On a back side of the slider 30, a fixed ring 32 is provided, through a coil spring (elastic member) 31.

Further, a base end portion of a holding part 33 is turnably connected to a distal end portion of the operation pipe 27 through a working pin. The holding part 33 constitutes, together with a distal end part 41 of a probe 16, the treatment part of the ultrasonic medical device 10. When the operation pipe 27 is moved in the axial direction, the holding part 33 is pushed/pulled in the front/back direction through the working pin. At this time, when the operation pipe 27 is moved to an operator's hand side, the holding part 33 is turned about a fulcrum pin in a counterclockwise direction through the working pin. As a result, the holding part 33 is turned in a direction approaching the distal end part 41 of the probe 16 (closing direction). At this time, a body tissue can be held between the cantilever holding part 33 and the distal end part 41 of the probe 16.

In a state where the body tissue is thus held, an electric power is supplied from an ultrasonic power supply to the ultrasonic transducer 1 to vibrate the ultrasonic transducer 1. This ultrasonic vibration is transmitted to the distal end part 41 of the probe 16. Then, the ultrasonic vibration is used to treat the body tissue held between the holding part 33 and the distal end part 41 of the probe 16.

As illustrated in FIG. 9, the transducer unit 13 is a unit obtained by integrally assembling the ultrasonic transducer 1 and the probe 16 which is a rod-like vibration transmission member that transmits the ultrasonic vibration generated in the ultrasonic transducer 1.

A horn 42 that amplifies an amplitude of the ultrasonic vibration is connected to the ultrasonic transducer 1. The horn 42 is formed of duralumin, stainless steel, or a titanium alloy such as 64Ti (Ti-6Al-4V). The horn 42 is formed into a cone shape having an outer diameter reduced toward a distal end thereof and has an outward flange 43 on a base end outer peripheral portion thereof. The shape of the horn 42 is not limited to the cone shape, but may be an exponential shape having an outer diameter exponentially reduced toward the distal end thereof or a step shape having an outer diameter reduced stepwise toward the distal end thereof.

The probe 16 has a probe main body 44 formed of a titanium alloy such as 64Ti (Ti-6Al-4V). On a distal end side of the probe main body 44, the ultrasonic transducer 1 connected to the horn 42 is provided. In such a manner as described above, the transducer unit 13 integrally including the probe 16 and ultrasonic transducer 1 is formed. In the probe 16, the probe main body 44 and horn 42 are threadably connected to each other, and the probe main body 44 and horn 42 are screwed to each other.

The ultrasonic vibration generated in the ultrasonic transducer 1 is amplified by the horn 42 and is then transmitted to the distal end part 41 of the probe 16. A treatment part to be described later for treating the body tissue is formed at the distal end part 41 of the probe 16.

Further, on an outer peripheral surface of the probe main body 44, two ring-shaped rubber linings 45 formed of an elastic member are fitted to several locations of a vibration node position, which is on the midway in the axial direction of the probe main body 44, so as to be spaced apart from each other. These rubber linings 45 prevent contact between the outer peripheral surface of the probe main body 44 and the operation pipe 27 to be described later. That is, in the course of the assembly of the insertion sheath part 18, the probe 16 as a transducer-integrated probe is inserted inside the operation pipe 27. At this time, the rubber linings 45 prevent contact between the outer peripheral surface of the probe main body 44 and the operation pipe 27.

Further, the ultrasonic transducer 1 is electrically connected, through an electric cable 46, to an unillustrated power supply device body that supplies current for use in generating the ultrasonic vibration. Supplying electric power from the power supply device body to the ultrasonic transducer 1 through wiring in the electric cable allows the ultrasonic transducer 1 to be driven. The transducer unit 13 includes the ultrasonic transducer 1 that generates the ultrasonic vibration, the horn 42 that amplifies the generated ultrasonic vibration, and the probe 16 that transmits the amplified ultrasonic vibration.

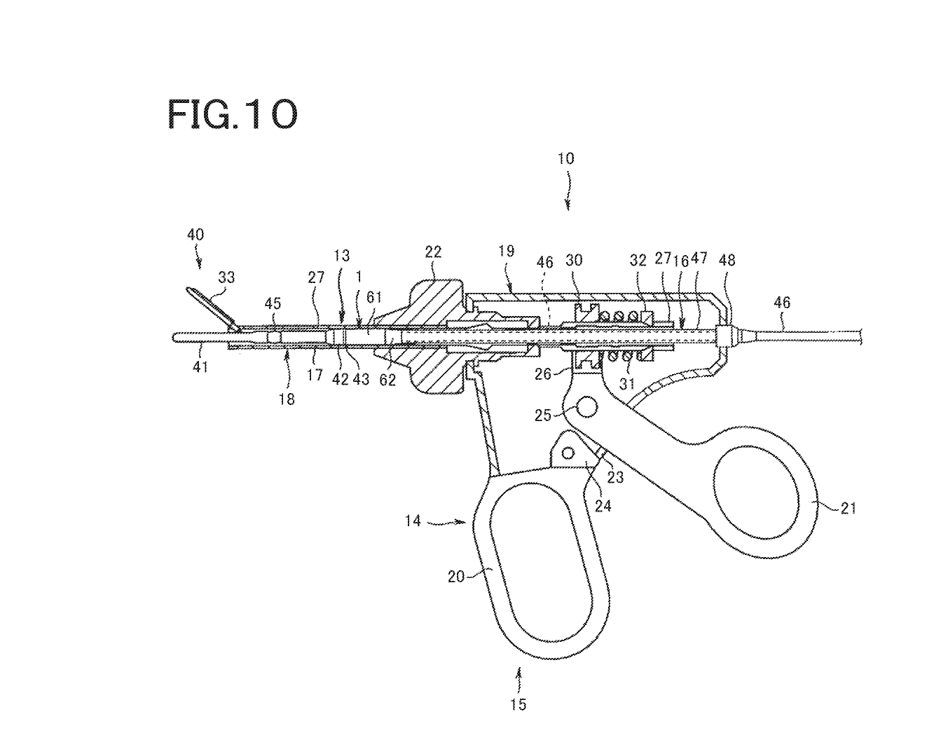

FIG. 10 illustrates an entire configuration of an ultrasonic medical device according to another aspect of the ultrasonic medical device according to the present embodiment.

The ultrasonic transducer 1 and transducer unit 13 need not be housed inside the operation part main body 19 as illustrated in FIG. 8, but may be housed inside the operation pipe 27 as illustrated in FIG. 10. In the ultrasonic medical device 10 of FIG. 10, the electric cable 46 extending between a bending stopper 62 of the ultrasonic transducer 1 and a connector 48 provided at a base portion of the operation part main body 19 is inserted through a metal pipe 47 and housed therein. The connector 48 is not essential, but, instead, a configuration may be adopted in which the electric cable 46 is extended up to the inside of the operation part main body 19 and is connected to the bending stopper 62 of the ultrasonic transducer 1. The configuration of the ultrasonic medical device 10 as illustrated in FIG. 10 can further save the interior space of the operation part main body 19. The function of the ultrasonic medical device 10 of FIG. 10 is the same as that of the ultrasonic medical device 10 of FIG. 8, so detailed descriptions thereof will be omitted.

As described above, the ultrasonic transducer 1 according to the present embodiment includes the two metal blocks 2, the plurality of piezoelectric elements 3 stacked between the metal blocks 2, the bonding material 4 bonding the metal block 2 and piezoelectric element 3, and the piezoelectric elements 3 to each other, and the heterogeneous material part 5 provided in the notch part 2b formed in the bonding plane 2a of the metal block 2 with respect to the piezoelectric element 3 and having a thermal expansion coefficient different from that of the metal block 2. With this configuration, there can be provided an ultrasonic transducer 1 with a reduced stress and an excellent vibration transmission efficiency.

Further, in the ultrasonic transducer 1 according to the present embodiment, the heterogeneous material part 5 is provided in the notch part 2b formed in an outer periphery of the metal block 2, thus facilitating the formation thereof.

Further, in the ultrasonic transducer 1 according to the present embodiment, assuming that a thermal expansion coefficient of the metal block 2 is α2 and that a thermal expansion coefficient of the heterogeneous material part 5 is α5, at least a relationship of α5<α2 is satisfied, allowing further stress reduction.

Further, in the ultrasonic transducer 1 according to the present embodiment, assuming that a predetermined one direction on the bonding plane 2a is x and that a direction perpendicular to x is y, when a thermal expansion coefficient α3x of the piezoelectric element 3 in the x-direction and a thermal expansion coefficient α3y thereof in the y-direction have a relationship of α3x>α3y, a relationship among x- and y-direction-dimensions 5x and 5y from the outer periphery of the metal block 2 to the heterogeneous material part 5 and x- and y-direction-dimensions 2x and 2y of the metal block satisfy (5x/2x)<(5y/2y), thereby allowing stresses reduction in accordance with the anisotropy of the thermal expansion coefficient of the piezoelectric element 3.

Further, in the ultrasonic transducer 1 according to the present embodiment, the heterogeneous material part 5 is provided in the notch part 2b formed inside the metal block 2, thus facilitating the formation thereof.

Further, in the ultrasonic transducer 1 according to the present embodiment, assuming that a thermal expansion coefficient of the metal block 2 is α2, that a thermal expansion coefficient of the piezoelectric element 3 is α3, and that a thermal expansion coefficient of the heterogeneous material part 5 is α5, at least a relationship of α2<α5 is satisfied, allowing further stress reduction.

Further, in the ultrasonic transducer 1 according to the present embodiment, assuming that a predetermined one direction on the bonding plane 2a is x and that a direction perpendicular to x is y, when a thermal expansion coefficient α3x of the piezoelectric element 3 in the x-direction and a thermal expansion coefficient α3y thereof in the y-direction have a relationship of α3x>α3y, a relationship among x- and y-direction-dimensions 5x and 5y from the outer periphery of the metal block 2 to the heterogeneous material part 5 and x- and y-direction-dimensions 2x and 2y of the metal block satisfy (5x/2x)<(5y/2y), thereby allowing stresses reduction in accordance with the anisotropy of the thermal expansion coefficient of the piezoelectric element 3.

Further, the ultrasonic medical device 10 according to the present embodiment includes the ultrasonic transducer 1 and a probe distal end part receiving the ultrasonic vibration generated in the ultrasonic transducer 1 and treating the body tissue. Thus, there can be provided an ultrasonic medical device 10 with a reduced stress and an excellent vibration transmission efficiency.

The present invention is not limited to the above embodiments. That is, in describing the embodiments, many specific details are included for illustrative purpose; however, a person skilled in the art can understand that the details added with variations or modifications do not exceed the scope of the present invention. Therefore, the illustrative embodiments of the present invention have been described without causing the claimed invention to lose generality and without imposing any limitation thereon.

For example, although in the ultrasonic transducer 1 according to the present embodiment, the metal block 2 and piezoelectric element 3 are each formed into a rectangular parallelepiped shape, they may be formed into a cube or a column. Further, the heterogeneous material part 5 may be formed so as to match with the cross-sectional shapes of the metal block 2 and piezoelectric elements 3, or may be formed into a shape different therefrom, as illustrated in FIGS. 4B and 7B.

### REFERENCE SIGNS LIST

1: Ultrasonic transducer

2: Metal Block

3: Piezoelectric element

4: Bonding material

5: Heterogeneous material part

1. An ultrasonic transducer comprising:
two metal blocks;
a plurality of piezoelectric elements stacked between the metal blocks;
a bonding material bonding the metal blocks and piezoelectric elements, and the piezoelectric elements to each other, and
a heterogeneous material part provided in a notch part formed in a bonding plane of the metal block with respect to the piezoelectric element and having a thermal expansion coefficient differed from a thermal expansion coefficient of the metal block.

2. The ultrasonic transducer according to claim 1, wherein
the heterogeneous material part is provided in the notch part formed in an outer periphery of the metal block.

3. The ultrasonic transducer according to claim 2, wherein
assuming that a thermal expansion coefficient of the metal block is α2 and that a thermal expansion coefficient of the heterogeneous material part is α5, at least a relationship of α5<α2 is satisfied.

4. The ultrasonic transducer according to claim 1, wherein assuming that a predetermined one direction on the bonding plane is x and that a direction perpendicular to x is y, when a thermal expansion coefficient α3x of the piezoelectric element in the x-direction and a thermal expansion coefficient α3y thereof in the y-direction have a relationship of α3x>α3y, a relationship among x- and y-direction-dimensions 5x and 5y from the outer periphery of the metal block to the heterogeneous material part and x- and y-direction-dimensions 2x and 2y of the metal block satisfy (5x/2x)<(5y/2y).

5. The ultrasonic transducer according to claim 1, wherein the heterogeneous material part is provided in the notch part formed inside the metal block.

6. The ultrasonic transducer according to claim 5, wherein assuming that a thermal expansion coefficient of the metal block is α2, and that a thermal expansion coefficient of the heterogeneity material part is α5, at least a relationship of α2<α5 is satisfied.

7. The ultrasonic transducer according to claim 6, wherein assuming that a predetermined one direction on the bonding plane is x and that a direction perpendicular to x is y, when a thermal expansion coefficient α3x of the piezoelectric element in the x-direction and a thermal expansion coefficient α3y thereof in the y-direction have a relationship of α3x>α3y, a relationship among x- and y-direction-dimensions 5x and 5y from the outer periphery of the metal block to the heterogeneous material part and x- and y-direction-dimensions 2x and 2y of the metal block satisfy (5x/2x)<(5y/2y).

8. An ultrasonic medical device comprising:
an ultrasonic transducer as claimed in claim 1; and
a probe distal end part receiving ultrasonic vibration generated in the ultrasonic transducer and treating the body tissue.
